(19) United States
(12) Patent Application Publication
(10) Pub. No.: US 2003/0118560 A1
(43) Pub. Date: Jun. 26, 2003

(54) Composite biocompatible matrices

(76) Inventors: Sheila J. Kelly, Ramsey, MN (US); Matthew F. Ogle, Oronoco, MN (US)

Correspondence Address: ALTERA LAW GROUP, LLC, 6500 CITY WEST PARKWAY, SUITE 100, MINNEAPOLIS, MN, 55344-7704, US

(21) Appl. No.: 10/027,464

(22) Filed: Dec. 20, 2001

(51) Int. Cl. A61K 45/00; A61F 2/00

(52) U.S. Cl. 424/93.7; 424/424

(57) ABSTRACT

A composite matrix has a first layer and a second layer. The first layer includes at least about 5 dry weight percent flexibility modifying agent. The second layer includes at least about 5 dry weight percent less flexibility modifying agent than the first layer. A valved prosthesis with flexible leaflets supported by a wall can be formed from the composite materials. In some embodiments, the wall of the prosthesis includes a first layer with at least about 60 weight percent collagen and a second layer with at least about 25 weight percent collagen and at least about 5 weight percent elastin. The leaflets preferably include a first layer with at least about 60 weight percent collagen and a second layer with at least about 25 weight percent collagen and at least about 5 weight percent proteoglycans.

### BACKGROUND OF THE INVENTION

The invention relates to materials that mimic the mechanical properties of selected tissues and may be suitable for cell colonization in vivo and/or in vitro In particular, the invention relates to composite materials that impart desired mechanical properties as well as, in some embodiments, forming a suitable material for cell colonization of implantable medical articles.

Various medical articles have been designed particularly for contact with a patient's body fluids. This contact can be sufficiently long such that surface interactions between the medical article and the patient's blood and/or tissue become significant. For example, the interaction of blood with the surface of the medical article can lead to degradation, such as calcification of the medical article. Relevant medical articles include, for example, catheters and prostheses.

Prostheses, i.e., prosthetic devices, are used to repair or replace damaged or diseased organs, tissues and other structures in humans and animals. Prostheses must be generally biocompatible since they are typically implanted for extended periods of time. Prostheses can be constructed from natural materials, synthetic materials or a combination thereof.

Bioprosthetic heart valves from natural materials were introduced in the early 1960's. Bioprosthetic heart valves typically are derived from pig aortic valves or are manufactured from other biological materials such as bovine pericardium. Xenograft heart valves are typically fixed with glutaraldehyde prior to implantation to reduce the possibility of immunological rejection. Glutaraldehyde reacts to form covalent bonds with free functional groups in proteins, thereby chemically crosslinking nearby proteins.

The importance of bioprosthetic animal heart valves as replacements for damaged human heart valves has resulted in a considerable amount of interest in the long term performance of these valves and, in particular, in the affects of calcification on tissue-based transplants. Calcification, i.e., the deposit of calcium salts, especially calcium phosphate (hydroxyapatite), can occur in and on some materials of a medical article while contacting the patient's body fluids. Calcification can affect the performance and structural integrity of medical articles constructed from these calcification sensitive materials, especially over extended periods of time. For example, calcification is the primary cause of clinical failure of bioprosthetic heart valves made from porcine aortic valves or bovine pericardium. Calcification is particularly severe at stress points where suture passes through tissue.

Generally, few bioprosthetic valves remain functional after 20 years. Replacement of a degenerating valve prosthesis subjects the patient to additional surgical risk, especially in the elderly and in situations of emergency replacement. While failure of bioprostheses is a problem for patients of all ages, it is particularly pronounced in younger patients. Over fifty percent of bioprosthetic valve replacements in patients under the age of 15 fail in less than five years because of calcification. Other prostheses made from natural and/or synthetic materials also display clinically significant calcification.

As a result, there is considerable interest in preventing the deposit of calcium on implanted biomaterials, especially heart valves. Research on the prevention of calcification has focused to a considerable extent on the pretreatment of the biomaterial prior to implantation. A significant advance toward reducing calcification of bioprostheses was the determination that Al⁺³ cations and other multivalent cations inhibit calcification.

Other approaches to reducing calcification have employed a chemical process in which at least some of the reactive glutaraldehyde moieties are inactivated. Still other approaches have included development of alternative fixation techniques, since evidence suggests that the fixation process itself contributes to calcification and the corresponding mechanical deterioration. In addition, since nonviable cells present in transplanted tissue are sites for calcium deposition, various processes have been developed to remove cellular material from the collagen—elastin matrix of the tissue prior to implantation.

Another disadvantage of tissue-based prostheses is the failure of such devices to be self maintaining. Long term durability is enhanced by the ability of viable cells to populate the implanted tissue and to carry out maintenance functions. The importance of viable cells has been studied in the context of homograft transplants, i.e., transplants from one member of a species to another member of the same species. Proper homograft preservation can maximize the number of viable cells remaining in the tissue as determined by matrix protein synthesis. Preservation techniques that do not promote cell survival, such as long-term storage at 4° C., are associated with reduced in vivo durability. Even following preservation techniques, difficulties are presented by potential rejection of the tissue and by maintenance of the tissue with viable cells prior to implantation.

Thus, cell ingrowth into prosthetic tissue material can decrease the prevalence of calcification and reintroduce some degree of self maintenance similar to a native valve. However, substrates for the formation of biosynthetic tissue may not have desired mechanical/structural properties for longer term improved performance in heart valves or may not provide desirable characteristics for cell colonization.

### SUMMARY OF THE INVENTION

In a first aspect, the invention pertains to a composite matrix including a first layer and a second layer. The first layer has at least about 5 dry weight percent flexibility modifying agent. The second layer has at least about 5 dry weight percent less flexibility modifying agent than the first layer. At least one layer comprises a reconstituted composition.

In a further aspect, the invention pertains to a valved prosthesis including a wall and a plurality of flexible leaflets supported by the wall. The wall includes a composite matrix having a first layer and a second layer. In the composite matrix of the wall, the first layer has at least about 60 dry weight percent collagen, and the second layer has at least about 25 dry weight percent collagen and at least about 5 dry weight percent elastin. The leaflets include a composite matrix having a first layer and a second layer. In the composite matrix of the leaflets, the first layer has at least about 60 dry weight percent collagen, and the second layer has at least about 25 dry weight percent collagen and at least about 5 dry weight percent proteoglycans.

In another aspect, the invention pertains to a method of forming a composite matrix. The method includes the step of fastening a first layer with a second layer. The first layer includes at least about 25 dry weight percent collagen. The second layer includes a flexibility modifying bio-macromolecule.

### BRIEF DESCRIPTION OF THE DRAWINGS

FIG. 1 is a side perspective view of a multiple leaflet heart valve prosthesis.

FIG. 2 is a side perspective view of a multiple leaflet mitral heart valve prosthesis.

FIG. 3A is a perspective view of a vascular prosthesis.

FIG. 3B is a side view of the vascular prosthesis of FIG. 3A attached to blood vessels.

FIG. 4 is a fragmentary side view of a two-layer composite matrix.

FIG. 5 is a fragmentary side view of a three layer composite matrix.

FIG. 6 is a fragmentary side view of a five layer composite matrix.

FIG. 7 is a fragmentary side view of a composite matrix with an outer layer folded over an inner layer.

FIG. 8 is a first composite matrix component of the heart valve prosthesis of FIG. 1.

FIG. 9 is a second composite matrix component of the heart valve prosthesis of FIG. 1.

FIG. 10 is a third composite matrix component of the heart valve prosthesis of FIG. 1.

FIG. 11 is a fourth composite matrix component of the heart valve prosthesis of FIG. 1.

FIG. 12 is a leaflet segment component of the aortic heart valve prosthesis of FIG. 2.

FIG. 13 is a post segment that reinforces the commissure posts of the aortic heart valve prosthesis of FIG. 2.

FIG. 14 is a rim strip that connects with leaflets along scallops and with the commissure posts of the aortic heart valve prosthesis of FIG. 2.

FIG. 15 is a micrograph of a composite matrix formed with bovine pericardium and elastin.

FIG. 16 is a micrograph of a composite matrix formed with bovine pericardium and hyaluronic acid.

FIG. 17 is a micrograph of a composite matrix formed with bovine pericardium and a blend of elastin and collagen, in which the section is perpendicular to aligned elastin fibril.

FIG. 18 is a micrograph of the composite matrix of FIG. 17, in which the section is taken along the length of the elastin fibrils.

FIG. 19 is a micrograph at 4× of a composite matrix formed with bovine pericardium and a blend of hyaluronic acid and collagen.

FIG. 20 is a micrograph of the composite matrix of FIG. 19 at 10×.

### DETAILED DESCRIPTION OF THE ILLUSTRATIVE EMBODIMENTS

A composite material has been developed as a matrix for a biosynthetic tissue that incorporates a plurality of layers that impart desirable mechanical properties with one or more of the layers having desirable properties for cell colonization. Furthermore, the composite material can be formed in layers such that the interaction of the layers reduces friction and wear to enhance long term durability and maintain mechanical properties. In some embodiments, a surface material selected for cell colonization is folded over or sandwiched around an internal material with desirable mechanical properties. In other embodiments, one or more layers prepared for cell colonization are colonized by cells in vitro and placed within other layers that may or may not be prepared for cell colonization to form the composite. In various embodiments, a material selected for its mechanical properties can incorporate, for example, proteoglycans for lubricating properties and flexibility and/or elastin or other proteins for flexibility.

The composite with one or more layers prepared for cell colonization can be modified with compounds, such as growth factors, cytokines and the like, and/or with texturing, such as porosity, to encourage cell colonization. The use of layers with different chemical compositions can impart desirable mechanical properties to the composite, such as high strength and durability along with desired flexibility and reduction of internal stress. The composite material can be advantageously used as a biosynthetic tissue matrix in the formation of valved prostheses, especially heart valve prostheses.

In general, relevant medical devices are bioprostheses that are formed to mimic a corresponding structure within the body, although suitable medical devices can be percutaneous devices with long term contact with body fluids. The bioprostheses can be used to replace the corresponding native structure. The medical devices can be prosthetic devices suitable for long term implantation within a recipient patient. Generally, the patient is an animal, preferably a mammal, such as a human.

The medical devices include at least a component that preferably is formed from a composite matrix that forms a biosynthetic tissue following colonization of the composite matrix by viable cells. A component of the medical device with a composite matrix can have specific mechanical requirements for preferred function within the medical device.

It is desirable to form medical devices, especially implantable prostheses, from materials that are long lasting and mimic natural function following implantation. Tissue has many desirable properties that make it a preferred material for many implantable prostheses. Fixed tissue has found valuable use in implantable prostheses, but due to the lack of viable cells, traditional fixed tissue tends to calcify and otherwise change in undesirable ways following extended periods of implantation.

For blood contacting prostheses, tissue with viable cells are especially blood compatible and non-thrombogenic. However, maintenance and long term storage of tissue with viable cells can be problematic, and autograft tissue that is compatible with the patient may be difficult to obtain. Therefore, it is desirable to develop tissue matrices suitable for colonization using viable cells, either in vivo or in vitro, for incorporation into medical devices. The cells can be the patient's own cells to reduce rejection issues, although other cell sources are available for in vitro cell colonization.

The formation of suitable composite matrices for cell colonization preferably takes into account several factors. First, the materials in the composite matrices should be non-toxic to cells such that cells can proliferate when in contact with the matrix. In particularly preferred embodiments, the matrix or portion thereof would encourage the proliferation of cells in contact with the matrix or even attract cells to the tissue matrix. In addition, the composite matrix preferably has suitable mechanical properties for the intended purpose of the material. The composite matrix should be stable with time such that the matrix has acceptable properties initially and after passage of an extended period following implantation. Cell colonization can help stabilize the composite matrix following implantation if fibroblast cells colonize the matrix since these cells produce collagen and other extracellular matrix materials to generate, repair and replace the extracellular matrix. Cell colonization by endothelial cells may improve hemodynamic performance and reduce valve complications.

Composite matrices described herein include a composite with a plurality of layers having different compositions. The layers are joined together to form an integrated composite. However, the layers may be but are not necessarily formed from a uniform material throughout the layer. In addition, the layers may not have a sharp boundary separating the layers.

The final composite can have two, three or more than three layers. Surface layers preferably are suitable for cell colonization. Generally, layers have different mechanical properties from other layers within the composite. The tissue matrices described herein can be produced with high reproducibility, with desirable mechanical properties, in forms that are highly suitable for cell colonization and with a good durability for long-term use without undesirable wear.

A variety of materials can be used for the layers within the composite. Suitable materials for the formation of biosynthetic tissue matrices or layers thereof generally include collagen, elastin and/or proteoglycans as a major component. Collagen is a significant component of the extracellular matrix of natural tissues. For particular tissues, such as the aortic wall, elastin can be a significant component. Proteoglycans can impart desired flexibility and friction reduction.

Natural tissues are suitable candidates as materials for the formation of tissue matrices. In particular, natural tissues can be used as a layer in the composite, generally following treatment of the tissue, for example, by crosslinking and/or chemical treatment. However, tissue materials may have disadvantages due to variability. In addition, natural tissue materials may not have desired levels of mechanical durability or may not have appropriate mechanical properties or compositions for specific prostheses. The composites described herein can make up for some of the deficiencies of tissues alone as matrix materials. In addition, some of the other materials used as composite layers may include compositions similar to natural tissues with modified concentrations to provide specific desired properties. The composite matrices herein generally can be formed consistently with desired properties.

Collagen, elastin and/or proteoglycans obtained from tissue or other sources can be combined with other components to form a layer of material for incorporation into the composite. Materials for a particular layer are selected for the desired properties for the particular layer.

Surface layers and possibly other layers of the composite are generally suitable for cell colonization. Suitable matrix layers can be derived from mammalian tissue sources, such as pericardium tissue, for example bovine pericardium, fascia and intestinal collagen material (ICL). Due to its high uniformity, intestinal collagen materials are preferred materials for use as durable, less flexible layers, especially for cell colonization. Generally, the surface material includes compositions, such as growth factors or the like, that encourage cell colonization of the material.

Additional layers can be selected based on their mechanical properties. For many vascular applications, the prosthesis material is preferably somewhat flexible. Flexibility facilitates function as well as offering the potential to improve longevity by reducing stresses in response to strain forces. Layers of the composite can include components that reduce friction within the layer and between the layer and adjacent layers. Flexibility modifying agents can be, for example, bio-macromolecules, such as elastin and proteoglycans, or synthetic polymers.

Elastin is a natural protein that can impart flexibility to a collagen based material. Elastin is found naturally in certain tissues as part of the extracellular matrix. Proteoglycans are protein/carbohydrate composites that are useful in reducing friction and, therefore, stresses within the material. Proteoglycans are found in the extracellular matrix of certain tissue, such as heart valve leaflets and cartilage.

In some preferred embodiments, the composite matrix includes a colonization layer and a flexible layer, although the flexible layer can also be prepared to encourage cell colonization. The colonization layer can be mostly collagen that may have been modified with compositions that promote cell colonization. The flexible layer can include, for example, a flexibility modifying bio-macromolecule, such as elastin, proteoglycans or a mixture thereof. In some embodiments, the flexible layer is surrounded on both sides with a colonization layer, although the flexible layer can be suitable for cell colonization also. Specifically, for example, a flexible layer can be placed between two layers of colonization layer material, or a larger layer of colonization material can be folded over a flexible layer.

The composite materials are especially suitable for the formation of heart valve prostheses. Different composite materials can be selected for the formation of the prosthesis wall and the prosthesis leaflets. A natural heart valve wall has collagen and significant amounts of elastin. The composite used to form the wall of the prosthesis can include a layer of collagen/elastin material within cell colonization layers of collagen. A natural heart valve leaflet includes collagen and proteoglyeans. Thus, a composite used to form the leaflets of the prosthesis can include a layer of collagen with proteoglycans within cell colonization layers of collagen.

Thus, a heart valve prosthesis can be constructed from composite materials that mimic the natural materials in the heart valve. While the synthetic materials have structural differences from the native materials, the synthetic materials can be constructed to imitate the mechanical properties of the natural valve. While a natural valve could be used to form a tissue matrix by decellularization and/or crosslinking, processing of the natural tissue can degrade the compositional and mechanical properties. Also, the synthetic matrix material formed from the composites are highly reproducible, which increases yields and provides for more satisfactory performance expectations.

Suitable materials are formed into the individual layers. For example, an intestinal collagen material can be infused with appropriate compounds to facilitate cell colonization. A flexible layer can be constructed by forming a mixture of solubilized collagen from a suitable source and the selected flexibilizing composition. After the mixture is properly combined, the solvent is removed, and the resulting material is formed into a layer of appropriate dimensions.

The layers are then fused together to form the composite. The fusing of the layers can be accomplished by various approaches. For example, the layers can be fused by an adhesive, such as a biologic glue. Alternatively, a crosslinking agent can be used to fuse the layers. In other embodiments, the layers are fused with pressure and/or heat. A combination of these approaches can be used to fuse the layers with desired degrees of shear strength.

The composite materials are then assembled into the desired prosthesis. The composites may be cut to have a desired size and shape. The components then can be attached together to form the prosthesis. The prosthesis may or may not include stents, sewing cuffs, or other frameworks to provide structural support or to facilitate implantation of the prosthesis.

The composites described herein can be used to form prostheses with superior performance. The particular materials can be selected for the particular application to have desired mechanical properties. A single prosthesis can include a plurality of different composites with each material having desirable properties for the particular location and function within the prosthesis. Due to the mechanical properties of the material and the strength resulting from the multiple layers, the materials can maintain suitable mechanical performance over long periods of use. In addition, the composites can have mechanical properties similar to the native structure being replaced.

Furthermore, by forming a composite with a chemical composition similar to the native tissue, the hydrophobic/hydrophilic nature and other physical/chemical characteristics of the material may more closely approximate the native tissue. For example, proteoglycans attract water into the material. The physical/chemical characteristics can affect calcium influx, cellular ingrowth and the like. Thus, having similar physical/chemical characteristics may improve the colonization of the composite matrix with appropriate cell types and cell densities such that the resulting biosynthetic tissue evolves into a material that more closely resembles the native tissue after cell colonization.

The composites provide the capability of combining various desirable features into a material. Increased durability can be obtained by the selection of components that resist delamination of the layers. Similarly, the mechanical properties of the composite can be selected appropriately for the use of the material. The ability to include one or more layers that encourage cell colonization can result in tissue that is self-repairing through the formation of extracellular matrix material by the cells. The combination of these capabilities introduces the capability to form a composite matrix that mimics the native matrix.

Medical Devices

Relevant medical devices generally include a composite matrix material. The composite matrix can be suitable for cellular attachment. Generally, these medical devices are prostheses or components designed for implantation into or onto a patient for extended periods of time. Prostheses include, for example, artificial hearts, artificial heart valves, annuloplasty rings, pericardial patches, vascular and structural stents, vascular grafts or conduits, pledgets, suture, permanently in-dwelling percutaneous devices, vascular shunts, dermal grafts for wound healing, and surgical patches. “Vascular” sites and structures as used herein include cardiovascular sites and structures and other blood contacting sites and structures. Biomedical devices that are designed to dwell for extended periods of time within a patient are also relevant for modification as described herein.

Particularly preferred medical devices include heart valve prostheses, vascular grafts, patches and dermal grafts. Heart valve prostheses can include a stent that serves as a frame for flexible leaflets, or the valve can be stentless, in which a heart valve is implanted utilizing the recipient's native support structure, i.e., the aorta or mitral annulus. The composite matrices can be incorporated into existing designs or new designs for medical devices assembled from tissue materials.

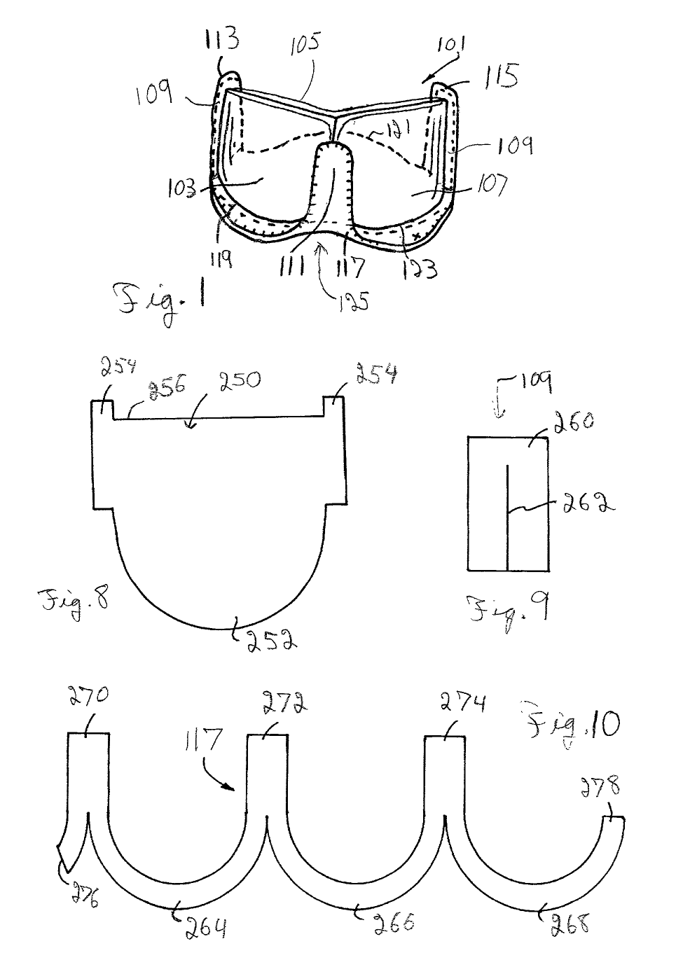

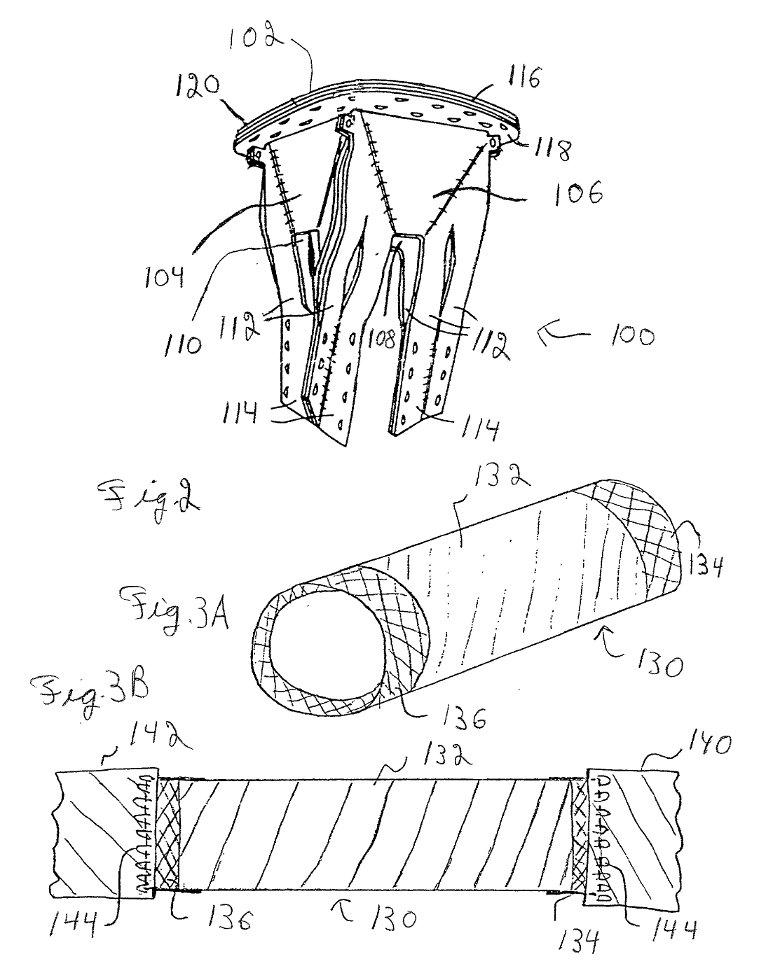

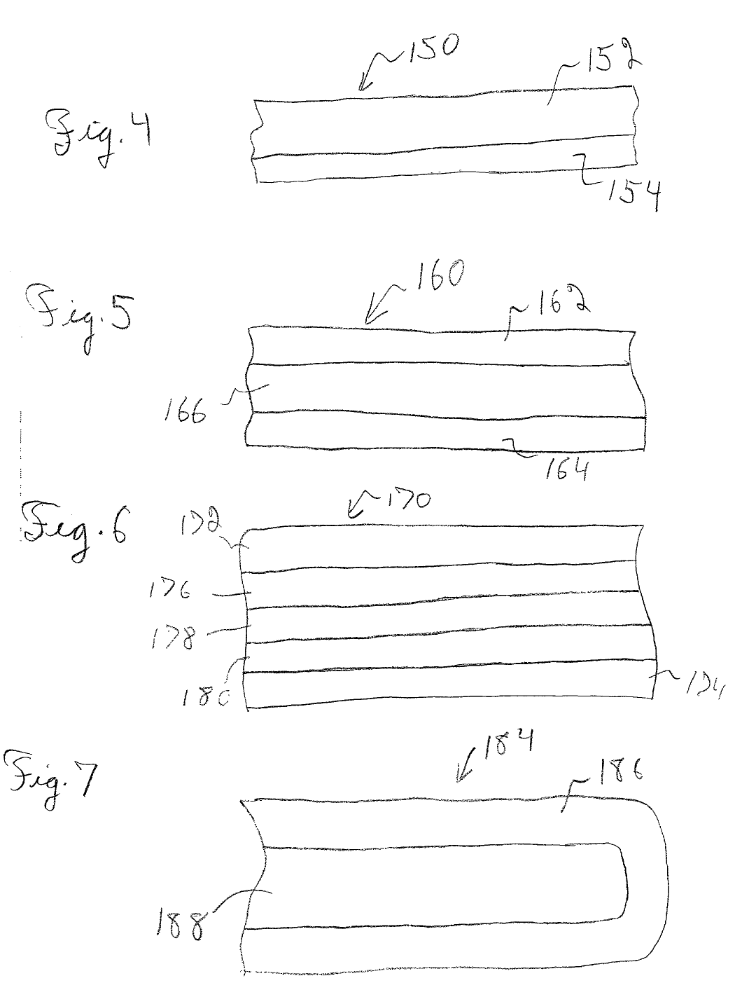

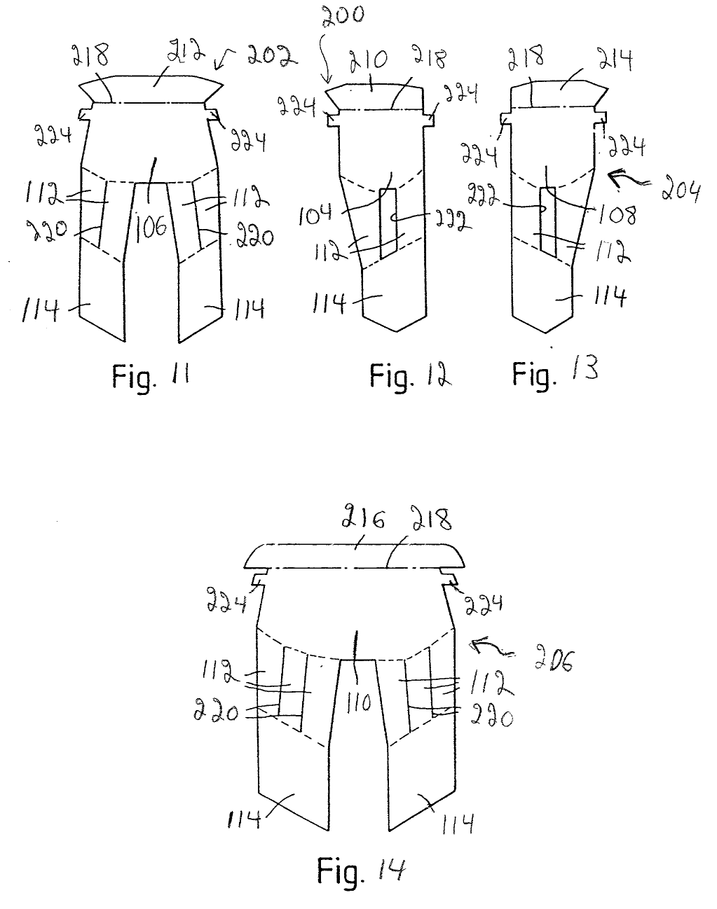

As a particular example of a heart valve prosthesis assembled from composite matrix elements, heart valve prosthesis 101 has three leaflets 103, 105, 107, as shown in FIG. 1. Leaflets 103, 105, 107 are attached to post segments 109 at commissure posts 111, 113, 115. A scalloped rim strip 117 forms a wall joining post segments 109 and leaflets 103, 105, 107 along scallops 119, 121, 123 to form a valve structure with an inflow edge 125 at scallops 119, 121, 123.

Another example of a heart valve prosthesis assembled from composite matrix elements is shown in FIG. 2. A stentless mitral heart valve prosthesis 100 with four leaflets includes a sewing ring 102, and four leaflets 104, 106, 108, 110. Chordae 112 extend from leaflets 104, 106, 108, 110. Chordae 112 and associated leaflets can be formed from a single sheet of biocompatible material, such as a composite matrix. Chordae 112 connect with attachment sections 114 for attachment to the patient's papillary muscles. An edge 116 of the composite matrix forming leaflets 104, 106, 108, 110 is stitched between two portions 118, 120 of sewing ring 102 to secure the leaflets to the sewing ring.

A representative vascular graft 130 is depicted in FIG. 3A. Vascular graft 130 includes a flexible tubular structure 132 and optional sewing cuffs 134, 136. Flexible tubular structure 132 can include one or more biocompatible materials, such as tissue, synthetic polymers or combinations thereof. Sewing cuffs 134, 136 are formed from fabric, tissue or the like. Sewing cuffs 134, 136 assist with the implantation of the prosthesis and may provide reinforcement of the prosthesis at the site of anastomoses, i.e., attachment of the vessel to the graft. A side view of vascular graft 130 attached to natural vessel sections 140, 142 is depicted in FIG. 3B. As shown in FIG. 3B, suture 144 is used to secure vascular graft 130 to vessel sections 140, 142, although other fastening approaches can be used.

The relevant medical devices include composite matrices, as described in the following section. The medical devices can also include other biocompatible materials, such as polymers, ceramics and metals. Appropriate ceramics include, without limitation, hydroxyapatite, alumina and pyrolytic carbon. Biocompatible metals include, for example, titanium, cobalt, stainless steel, nickel, iron alloys, cobalt alloys, such as Elgiloy®, a cobalt-chromium-nickel alloy, MP35N, a nickel-cobalt-chromium-molybdenum alloy, and Nitinol®, a nickel-titanium alloy.

Polymeric materials can be fabricated from synthetic polymers as well as purified biological polymers. Appropriate synthetic materials include hydrogels and other synthetic materials that cannot withstand severe dehydration. Suitable polymers include bioresorbable polymers that are gradually resorbed after implantation within a patient.

Appropriate synthetic polymers include, without limitation, polyamides (e.g., nylon), polyesters, polystyrenes, polyacrylates, vinyl polymers (e.g., polyethylene, polytetrafluoroethylene, polypropylene and polyvinyl chloride), polycarbonates, polyurethanes, poly dimethyl siloxanes, cellulose acetates, polymethyl methacrylates, ethylene vinyl acetates, polysulfones, nitrocelluloses and similar copolymers. Bioresorbable synthetic polymers can also be used such as dextran, hydroxyethyl starch, derivatives of gelatin, polyvinylpyrrolidone, polyvinyl alcohol, poly[N-(2-hydroxypropyl) methacrylamide], poly(hydroxy acids), poly(epsilon-caprolactone), polylactic acid, polyglycolic acid, poly(dimethyl glycolic acid), poly(hydroxy butyrate), and similar copolymers. These synthetic polymeric materials can be formed into fibers or yams and then can be woven or knitted into a mesh to form a matrix or substrate. Alternatively, the synthetic polymer materials can be molded or cast into appropriate forms.

Biological polymers can be naturally occurring or produced in vitro by fermentation and the like or by recombinant genetic engineering. Purified biological polymers can be appropriately formed into a substrate by techniques such as weaving, knitting, casting, molding, extrusion, cellular alignment and magnetic alignment. Suitable biological polymers include, without limitation, collagen, elastin, silk, keratin, gelatin, polyamino acids, polysaccharides (e.g., cellulose and starch) and copolymers thereof.

Composites

The composite matrix structures include two or more layers of different collagen-based, elastin-based or proteoglycan-based materials. The overall performance of the composite is improved due to the interaction of the multiple layers. A layer may or may not have a uniform composition through the layer. In addition, layers do not necessarily have a sharp boundary separating the layers. One or more of the layers generally include components that improve the elasticity of the layer or decrease the friction within the layer or between layers upon flexing. These other components can make up a significant portion of a layer's structure. Other layers can impart structure and durability to the composite. At least one of the layers of the composite includes a reconstituted composition. Reconstituted compositions involve mixtures of synthetic and/or purified materials, possibly blended with fragmented natural materials, to form a non-natural material for incorporation into the composite. Thus, the composites are distinguishable from native tissues that have layers.

The outer layers preferably are suitable for cell colonization, although internal layers can also be prepared for cell colonization such that cells secreting intracellular matrix proliferate throughout the composite. Layers suitable for cell colonization preferably are prepared with components to reduce or eliminate cytotoxicity and/or with compounds to encourage cell association with the matrix material. In addition to the materials described in this section, one or more of the layers can include viable cells associated with the layer by in vitro cell culturing.

A basic structure of a composite matrix of the invention is shown in FIG. 4. Composite matrix 150 has a first layer 152 and a second layer 154. First layer 152 and second layer 154 have different properties and generally have different compositions from each other. Since first layer 152 and second layer 154 form the opposite surfaces of the composite, both layers generally are suitable for cell colonization.

While the boundary between two layers may not be sharply defined due to some commingling of the materials at the interface, the layers are identifiable due to qualitatively different compositions that correspondingly modify the properties of that layer and possibly between layers. The definition of layers may be subjective in that the boundary between the layers may be conceptual rather than physical. The concept of layers represents the physical property of the composition changing through the thickness of the material. The change in composition can be abrupt at a physical boundary, continuous over a short distance due to some commingling at an approximate physical boundary or gradual due to a gradient in composition over a significant fraction of the thickness.

Layers can be defined by particular ranges in composition. The layers may or may not have flat/planar boundaries. These ranges in composition may or may not correspond to exact physical boundaries that exist in the composite. Thus, the composition at a particular location within the thickness of the material can be determined to evaluate its location within a particular layer. If a particular material has different average composition from the material adjacent the particular material and forms a reasonable percentage of the thickness of the total composite, the particular material can be considered a layer. Thus, intermixing regions at the boundary between two materials can form a separate layer if the composition varies sufficiently from the other layers and if the intermixing region has an appropriate thickness to be separately identified as such.

Based on a definition of layers relating to composition, an approximate boundary between layers can be estimated with sufficient precision to define a meaningful layer thickness. In addition, the layer of the composite material preferably has a thickness of at least about 25 microns, generally from about 50 microns to about 2 millimeters (mm) and in other embodiments from about 75 microns to about 1 millimeter. The thickness is an average thickness if there is some variation in the layer. However, in some embodiments, the layers do not have a uniform thickness through the material, and these ranges may be relevant separately for the portions with different thicknesses. While the composites comprise a layered structure, the composites can be attached to or otherwise associated with other materials that may have different structures.

A three layer composite matrix is shown in FIG. 5. Composite 160 has a first surface layer 162, a second surface layer 164 and an internal layer 166. First surface layer 162 and second surface layer 164 preferably are suitable for cell colonization. Internal layer 166 may or may not be suitable for cell colonization.

A five layer composite is shown in FIG. 6. Composite 170 includes a first surface layer 172, a second surface layer 174 and internal layers 176, 178, 180. The composite generally can include any number of layers greater than two. The number of layers in the composite is influenced by the desired properties and thickness of the composite matrix and by the corresponding properties of the individual layers.

Another embodiment of a composite matrix is shown in FIG. 7. Composite 184 has a surface layer 186 positioned around an internal layer 188. Additional folded layers can be included in the structure of FIG. 7 with the outer folded layer preferably being suitable for cell colonization. Similarly, additional layers can be added as internal layers within the structure of FIG. 7, as desired, with appropriate modification of the dimensions of the outer folded layer(s).

Generally, the materials forming the layers in the particular composite structure include amounts of collagen, elastin and/or proteoglycans, optionally, with a selected amount of additional compounds. The collagen in the materials provides a mechanical framework similar to the role of collagen as an extracellular matrix material in a natural tissue. Collagen, as used herein, includes chemically modified forms of collagen protein.

Flexibility modifying agents can be, for example, bio-macromolecules or synthetic polymers. Bio-macromolecules include, for example, elastin, proteoglycans and the like. Elastin provides a mechanical framework that is more elastic than collagen. Proteoglycans provide to a layer a flexible, low friction material. The amounts of collagen, elastin and proteoglycans are selected based on the desired mechanical properties of the particular layer material.

In addition to collagen, elastin and/or proteoglycans, the materials can include other compositional components including, for example, mechanical modifiers, cell colonization facilitators, crosslinking agents and other additives. The additional components can be proteins or other types of compounds. In general, proteins described herein can include polypeptides with one or more subunits and derivatives, such as polypeptides with carbohydrates, fatty acids or other adducts covalently or noncovalently attached to the polypeptide. The amount of additional compositional components depends on the purpose of the composition and the desired properties of the material.

Collagen can be obtained from natural sources or from recombinant DNA expression. Natural sources include a range of tissues, generally from mammals, such as human, bovine, porcine, seal or kangaroo. The selection of the particular tissue may depend on the desired layer properties. Similarly, the processing of the natural tissue depends on the source and the desired product material. The material can be processed from the tissue to leave the original tissue structure substantially intact with appropriate modifications to the tissue, such as cross linking and addition of selected compositions, or the collagen can be purified from the tissue source by dissolving the tissue and separating the collagen for further processing into the desired layer material.

Conventional tissues that have been processed substantially intact for fabrication into cardiac prostheses include, for example, mammalian fascia and pericardium, especially bovine pericardium. Heart valve prostheses and the like have been manufactured from sections of bovine pericardium that are stitched together to form the desired structure. However, bovine pericardium has the disadvantage that the material may not be highly uniform. An alternative source of tissue that is generally more uniform, at least in thickness, from batch-to-batch and within a batch is intestinal collagen obtained from the small intestine of a mammal.

The small intestine of vertebrates has several layers of structure with a large concentration of collagen. The collagen rich core layers include the tunica submucosa and the tunica mucosa. The tunica mucosa further includes several layers, such as the lamina muscularis mucosa, the stratum compactum and the lamina epithelialis mucosa. Materials comprising the tunica submucosa, the stratum compactum and the lamina muscularis mucosa have been suggested to be suitable graft material. These materials are described, for example, in U.S. Pat. No. 4,956,178 to Badylak et al., entitled “Tissue Graft Composition,” incorporated herein by reference. Also, the tunica submucosa alone has been described as suitable graft material, see U.S. Pat. No. 5,733,337 to Carr, Jr. et al., entitled “Tissue Repair Fabric,” incorporated herein by reference. The tunica submucosa can be separated from the other materials by squeezing the raw materials between opposing rollers. These intestinal collagen sources may be suitable sources of collagen-rich tissue material for the formation of a very strong and durable layer of a composite matrix. The intestinal collagen sources can be chemically modified before or after formation into the composite to impart desirable properties, such as improved cell colonization properties.

Purified commercial collagen is available from bovine and porcine sources from, for example, Collagen Corporation, Palo Alto, Calif. The commercial collagen can be solubilized for further processing.

In addition, human recombinant collagen is available. The process for forming recombinant collagen involves inserting the gene coding for the protein into an expression cassette that can be transferred into a host organism for the production of the protein. Proteins formed by recombinant genetic engineering include proteins with mutated, lengthened or shortened amino acid sequences that have suitable properties for incorporation into an extracellular matrix.

Some layers including collagen have at least about 25 dry weight percent collagen. Some relatively rigid layers can be formed with higher percentages of collagen. These relatively rigid layers generally have at least about 60 dry weight percent collagen, in some embodiments at least about 75 dry weight percent collagen and in other embodiments from about 85 dry weight percent collagen to about 100 dry weight percent collagen. A person of ordinary skill in the art will recognize that ranges within these explicit ranges are contemplated and are within the present disclosure. Generally, all of the materials described herein are hydrophilic with significant and varying amounts of water. Component weights are expressed as dry weights to eliminate these varying contributions of the water. Some natural tissues can be used as a material with high dry weight percents collagen.

As noted above, one or more layers of the composite matrix generally include an additional component to alter the mechanical properties of a layer. For example, a layer can include elastin and similar proteins to impart elasticity to the layer. Suitable proteins can be called elastic proteins. Elastin is a natural, fibrous protein in muscle tissue for imparting flexibility. The distribution of fiber lengths or molecular weight of the elastin can be selected to provide desired properties to the resulting composite layer. Elastin can be purchased from, for example, Elastin Products Co., Inc., Owensville, Mo. and Sigma Chemical Co., Saint Louis, Mo. Other natural proteins that are also suitable to provide flexibility for a layer include, for example, silk. Elastin in a layer is being used specifically for structural purposes. Therefore, an engineered protein that mimics the properties of elastin would also be suitable.

Flexible layers include, for example, elastic layers and friction reduced layers. The materials for incorporation into an elastic layer generally include an amount of collagen mixed with elastin and/or other elastic property modifiers. In preferred embodiments, the elastic matrix layers include from about 5 dry weight percent collagen to about 95 dry weight percent collagen, in other embodiments, from about 25 to about 75 dry weight percent collagen, and in further embodiments, from about 35 to about 60 dry weight percent collagen. These elastic matrix layers generally include, for example, from about 5 dry weight percent to about 95 dry weight percent elastic protein, in other embodiments from about 25 to about 75 dry weight percent elastic protein and in further embodiments from about 40 to about 65 dry weight percent elastin. A person of skill in the art will recognize that other ranges within these explicit ranges are also within the scope of the disclosure. Furthermore, contemplated elastic layers also can be formed from essentially 100 percent elastic proteins, although these materials may not have desired levels of structural integrity. The flexible layers generally include collagen to provide greater structural integrity. The elastic matrix layers can also include friction reducing materials, cell colonization facilitators and fillers.

Other layers include compositions that reduce friction and provide flexibility within the layer and between the layer and adjacent layers. These compositions may or may not also alter the elasticity of the material. Suitable friction reducing agents include, for example, proteoglycans. Proteoglycans are proteins with covalently attached glucosaminoglycan chains attached. Glucosaminoglycans are unbranched polysaccharides composed of repeating disaccharides. The glucosaminoglycans can make up a majority of the mass of the proteoglycans. Proteoglycans are naturally found in cartilage and other tissues. Alternatively, glucosaminoglycans can be mixed with collagen, or the glucosaminoglycans can be covalently bonded to collagen to form a synthetic glycoprotein. Other natural products suitable as friction reducing agents include, for example, other glycoproteins or polysaccharides, such as chondroitin sulfate and hyaluronic acid. Mixtures of friction reducing agents can also be used. Generally, the friction reducing agents are biological macromolecules and can include other proteins, such as silk.

The reduced friction matrix layers generally include from about 5 to about 100 dry weight percent friction reducing agent, in other embodiments from about 25 to about 90 dry weight percent friction reducing agent with appropriate amounts of collagen and/or other compounds. If a matrix layer includes both an elastic protein and a friction reducing macromolecule, the layer can include from about 5 to about 95 dry weight percent elastic protein and from about 5 to about 95 dry weight percent friction reducing macromolecule along with, optionally, from about 5 to about 90 dry weight percent collagen. A person of skill in the art will recognize that additional ranges within these explicit ranges are within the present disclosure. In addition, a matrix layer with friction reducing biological macromolecules with or without elastin proteins can include cell colonization facilitators, and other additional components/compounds.

Low friction synthetic polymers also can be mixed with collagen to form a low friction layer. Suitable low friction polymers include, for example, polytetrafluoroethylene. Other flexibility modifying synthetic polymers include hydrogels, which are hydrophilic polymers that do not dissolve in aqueous solutions. Hydrogels can absorb shock and impart flexibility. Suitable polymers for the formation of hydrogels include, for example, poly(ethylene glycol), poly(hydroxyethyl methacrylate), partially or fully hydrolyzed poly(vinyl alcohol), poly(vinyl pyrrolidone), poly(ethyloxazoline), poly(ethylene oxide)-co-poly(propylene oxide) block copolymers, poloxamines, polyacrylamide, hydroxypropylmethacrylate (HPMA), carboxymethyl cellulose, hydroxyethyl cellulose, methylhydroxypropyl cellulose, polysucrose, hyaluronic acid, chondroitin sulphate, dextran, sodium alginate, derivatives of alginate, chitosan, derivatives of chitosan and mixtures and copolymers thereof. Alginate and chitosan are natural polysaccharides that can be crosslinked with polyfunctional anions, such as dicarboxylic acids, sulphate ions and carbonate ions. More rigid synthetic polymers can be introduced to reduce flexibility of a layer, if desired.

The composite materials described herein have a great range of design capability since both the number of layers and composition of particular layers can be selected. Based on the disclosure herein, a person of ordinary skill in the art can select a number of layers and composition to achieve desired mechanical properties of the result ing composite. In general, having one or more layers with high collagen amounts can contribute durability and stability while flexibility modifying agents in other layers can contribute desired amounts of elasticity and friction reduction. Thus, a particular composite can be engineered with a desired balance of properties. Generally, adjacent layers can be identified as distinct layers by the presence of a difference of at least about 5 dry weight percent flexibility modifying agent, in other embodiments, at least about 10 dry weight percent flexibility modifying agent, and in further embodiments, at least about 15 dry weight percent difference in flexibility modifying agent. A person of ordinary skill in the art will recognize that additional ranges within these explicit ranges are contemplated and are within the present disclosure.

The layers incorporating a flexibility modifying agent and any other components can be formed by solubilizing a mixture of the proteins/macromolecules and any other components into a concentrated solution with appropriate amounts of the different components. The concentrated solution can be formed into a layer and the solvent removed, for example, by lyopholization or by air drying.

Collagen and other protein-containing materials are generally fixed/crosslinked to mechanically stabilize the material, reduce or eliminate proteolysis and to remove antigens from the material such that immune responses are reduced. However, crosslinking of collagen can result in cytotoxicity of the material. Thus, either a crosslinking approach should be chosen that introduces low levels of cytotoxicity or the materials following crosslinking should be treated to reduce cytotoxicity to acceptable levels.

Glutaraldehyde, formaldehyde or a combination thereof is typically used for fixation, but other fixatives can be used, such as epoxies and other difunctional aldehydes. Aldehyde functional groups are highly reactive with amine groups in proteins, such as collagen. Other preferred crosslinking agents include epoxyamines.

Dialdehydes, including glutaraldehyde, may be self-polymerizing. Improved crosslinking results may be obtained with moderate sized glutaraldehyde oligomers that more closely match the distance between collagen fibers. There are a variety of ways of adjusting the distribution of glutaraldehyde oligomers. For example, the concentration can be lowered to yield an equilibrium distribution with an increased number of smaller oligomers. Alternatively, a selectively permeable membrane can be used to exclude larger glutaraldehyde oligomers from the vicinity of the tissue to be crosslinked. Improved results obtained by crosslinking with a selectively permeable membrane is described further in U.S. Pat. No. 5,958,669 to Ogle et al., entitled “Tissue Fixation With Crosslinking Compounds,” incorporated herein by reference.

Tissue crosslinked with dialdehydes can be treated to remove the cytotoxicity. Preferred compositions for the treatment of aldehyde crosslinked tissue are described further in copending and commonly assigned U.S. patent application, Ser. No. 09/666,823 to Ashworth et al., entitled “Biocompatible Prosthetic Tissue,” incorporated herein by reference.

Furthermore, one or more layers of the composite can be formed to be porous to alter the mechanical properties and/or to facilitate cell colonization. Similarly, a material can be selected for incorporation into a composite based on natural porosity of the material. Porosity can be introduced into a material, for example, by forming the layer on a textured surface, by introducing soluble particles that are subsequently solubilized to leave voids in place of the particles, by microfabrication or by combinations thereof. Suitable soluble particles include, for example, resorbable polymers and ferric chloride (FeCl₃), which is soluble in water. Microfabrication involves, for example, some form of etching to introduce the desired porosity. A mask can be used to shield selected portions of the substrate such that the pores are formed at the un-shielded locations. Suitable etching approaches can involve, for example, reactive vapors that decompose material at the particular locations. Suitable reactive compounds include, for example, acidic compounds.

Compounds for Facilitating Cell Colonization

Colonization of the substrate can be facilitated by the incorporation of appropriate compounds into the substrate. Suitable colonization stimulating compounds can assist with cellular attachment or the compounds can stimulate cellular proliferation. In preferred embodiments, the compounds are selected based on the desired cell types for colonization of the composite matrix to form a biosynthetic tissue.

For some natural tissues, including heart valves, the underlying structure includes fibroblast cells within an extracellular matrix. The fibroblast cells produce and maintain the extracellular matrix. The surface of a vascular/cardiovascular tissue has a layer of endothelial cells a few cells thick. The endothelial cells provide desirable surface properties to the tissue for blood flow. Specifically, the endothelial cells form a blood contacting surface that is highly non-thrombogenic and blood compatible.

A composite matrix for the formation of biosynthetic vascular tissue can include compounds suitable to encourage colonization by fibroblast cells, endothelial cells and/or progenator cells. Colonization by both types of cells is particularly preferred since the fibroblast cells would help to maintain the integrity of the extracellular matrix following implantation of the biosynthetic tissue and the endothelial cells may improve hemodynamic performance and surface stability of the biosynthetic tissue. Other cell types, such as myofibroblasts and aortic smooth muscle cells, can be present to perform various functions.

It is desirable for the surface layers of the composite matrix to be suitable sites for in vitro and/or in vitro cell colonization. As described above, the matrix material can be prepared with little or no cytotoxicity. In addition, the matrix materials can be imparted with compounds that facilitate cell colonization. Preferred compounds include, for example, growth factors, chemotactic agents and cell attraction compounds. Preferred surface composite matrix layers include compounds to facilitate colonization with both fibroblast cells and endothelial cells.

To facilitate cell colonization, the composite matrix can be treated with specific compounds to stimulate association of desirable cells with the matrix. For example, the composite matrix can be associated with one or more growth factors, such as vascular endothelial growth factor (VEGF) and/or fibroblast growth factor, and/or compounds that attract cell precursors to the matrix, i.e., attraction compounds. The association of the composite matrix with the desired compounds can be performed before, during or after formation of the material into the matrix layer.

VEGF refers to a family of polypeptides that have been found to preferentially stimulate growth of vascular endothelial cells over other cells, such as smooth muscle cells. Several forms of VEGF have been identified. VEGF polypeptides generally have sequence homology with platelet-derived growth factor, which can alter the migration and proliferation of a variety of cell types. VEGF has also been referred to as vascular permeability factor. Human recombinant VEGF₁₆₅ is available commercially from R&D Systems, Minneapolis, Minn. The use of VEGF in the production of prostheses has been described further in copending and commonly assigned U.S. patent applications Ser. No. 09/014,087 to Carlyle et al., entitled “Prostheses With Associated Growth Factors,” and 09/186,810 to Carlyle et al., entitled “Prostheses With Associated Growth Factors,” both of which are incorporated herein by reference.

Fibroblast growth factors refer to a group of proteins that are characterized by the binding of heparin. These proteins have also been called heparin binding growth factors. These proteins strongly stimulate the proliferation of fibroblasts and possibly a variety of other cells of meodermal, ectodermal and endodermal origin. Particular fibroblast growth factors may have unique and/or overlapping function with other fibroblast growth factors. Several structurally related human fibroblast growth factors (FGF) have been cloned and sequenced, including basic FGF, acidic FGF, int 2-hst1/k-FGF, FGF-5, FGF-6, keratinocyte growth factor, AIGF (FGF-8), glia-activating factor and FGF-11. These are described further in U.S. Pat. No. 6,110,893 to Hu et al., entitled “Human Growth Factor 11,” incorporated herein by reference.

As noted above, the composite matrix can be associated with attraction compounds that attract desired precursor cells. Desirable precursor cells include both progenitor cells that can mature into fibroblasts or endothelial cells, and cells that can differentiate or transdifferentiate into fibroblasts or endothelial cells. Precursor cells circulate in a patient's blood stream. These precursor cells are thus available to colonize suitable blood contacting substrates, such as the composite tissue matrices described herein. Suitable precursor cells can be removed from the blood stream and associated with a matrix that serves as the foundation for a viable prosthetic tissue. To initiate the colonization by the precursor cells, an attraction compound can be associated with the composite matrix. Circulating precursor cells are removed from circulation by the attraction compound and become associated with the composite matrix.

Antibodies or fragments thereof directed against cell surface antigens can be used as attraction compounds. Similarly, natural ligands or portions thereof for cell surface receptors, e.g., on cell precursors, can be used to form attraction compounds. Attraction compounds can be ligands for cell surface receptors. Similarly, combinatorial chemistry approaches can be used to screen a set of putative ligands to find a ligand with a suitable binding affinity. In combinatorial chemistry approaches, a set of putative ligands are evaluated for effectiveness such that relevant structures within a ligand can be identified. The putative ligands can be selected based on chemical principles or by analogy with known binding molecules.

Fibroblast precursor cells include monocytes and macrophages. Monocytes and macrophages with a HLA-DR marker on their surfaces are capable of transdifferentiating into fibroblasts. This transdifferentiation is described in M. L. Labat et al., “Possible monocytic origin of chondrosarcoma: in vitro transdifferentiation of HLA-DR blood monocyte-like cells from a patient with chondrosarcoma, into neo-fibroblasts and chondrocyte-like cells,” Biomed. & Pharmacother 51: 79-93 (1997), incorporated herein by reference. The HLA-DR marker is a protein complex. In some embodiments of the invention, the HLA-DR marker is used to select for preferred precursor monocytes/macrophages.

Antibodies or ligands directed against the HLA-DR marker can serve as suitable attraction compounds to bind these monocytes/macrophages. The use of attraction compounds to associate precursor cells with a substrate is described further in copending and commonly assigned U.S. patent application Ser. No. 09/203,052 to Carlyle et al., entitled “Substrates For Forming Synthetic Tissue,” incorporated herein by reference.

Precursors for endothelial cells, called angioblasts, are also found in the blood stream. In some embodiments, ligands for the endothelial cell surface marker, endoglin, can be used as attraction compounds to select for preferred endothelial cell precursors.

The association of a colonization stimulating composition, e.g., a growth factor and/or an attraction compound, with a composite matrix layer may involve direct attachment, application of a coating, including an adhesive or binder, or chemical binding, involving a binding agent in addition to the attraction compound/response modifier.

Direct attachment entails combining the substrate with a solution of the colonization stimulating compound(s) without the use of an additional chemical binder. Direct attachment may involve reactions of the colonization stimulating composition(s) with reactive groups within the composite matrix material. With the use of an adhesive, the treatment compound(s) associates with the composite matrix or layer thereof due to incorporation of the colonization stimulating compound(s) into the structure of the cured adhesive. Preferred adhesives include, for example, biologic glues such as fibrin glue, and the like. Fibrin glue can be formed from the polymerization of fibrinogen and thrombin. Suitable fibrin glues are available from, for example, Immuno AG, Austria.

In other embodiments, the association of a colonization stimulating composition with the composite matrix involves chemical binding initiated by a selected chemical reagent, a chemical binding agent. In contrast to the use of an adhesive, chemical binding involves specific molecular interactions with compositions in the crosslinked tissue, rather than a collective adhesion. Chemical binding can involve covalent bonding, a plurality of noncovalent chemical interactions, or a combination of both covalent and noncovalent interactions. Noncovalent chemical interactions include hydrogen bonding, van der Waals interactions, and molecular rearrangements, which characterize specific binding interactions, such as antibody-antigen interactions, protein-receptor binding and enzyme-substrate associations. The chemical binding of the colonization stimulating compositions with the composite matrix can involve covalent bonding to the surface of the crosslinked tissue with reactive agents, such as crosslinking agents. Suitable crosslinking agents include, for example, glutaraldehyde, or multifunctional carboxylic acid compounds that bond with amine functional groups in proteins.

Other Treatment Compounds

The association of viable cells with a composite matrix tends to reduce or eliminate adverse metabolic activity, such as calcification. However, it may still be desirable to include compositions to inhibit calcification, either to supplement natural effects of viable cells and/or to provide protection before complete development of cells permeates the composite matrix. Thus, it may be desirable to include one or more anti-calcification agents within the composite matrix.

Generally, the calcification reducing agents would be contacted with the composite matrix following crosslinking. Suitable calcification reducing agents include detergents (e.g., sodium dodecyl sulfate), toluidine blue, diphosphonates, and multivalent cations, especially Al⁺³, Mg⁺² or Fe⁺³, or corresponding metals that can oxidize to form the multivalent metal cations. The effectiveness of AlCl₃ and FeCl₃ in reducing calcification of crosslinked tissue is described in U.S. Pat. No. 5,368,608 to Levy et al., entitled “Calcification-Resistant Materials and Methods of Making Same Through Use of Multivalent Cations,” incorporated herein by reference.

Formation of Composites

The layers of the composite are fastened together to form a coherent matrix. Following preparation of the individual layers of matrix, the layers are positioned to form the desired structure. Suitable exemplary structures were described above with respect to FIGS. 4-7. The properly positioned layers are bound using a selected approach that may involve, for example, adhesives, lyophilization, pressure, heat, chemical crosslinking and combinations thereof. The bonding method results in the formation of the coherent composite matrix in which the layers are secured to each other.

All the layers of the composite can be assembled simultaneously. Alternatively, one layer can be added to the composite structure at a time until the desired structure is complete. In addition, the layers can be assembled in groups, with the groups then assembled into the final composite.

Adhesives can be applied at the interface between two layers to join the layers. Preferred adhesives are not cytotoxic. A variety of surgical adhesives can be adapted for this purpose. Suitable adhesives include, for example, fibrin glues.

Also, if the layers are placed in physical contact, the positioned layers can be lyophilized to join the layers. Lyophilization can be performed with commercially available equipment. Following lyophilization, the layers are physically joined. This adhesion of the layers is maintained to some degree even if the dried structure is rehydrated.

Pressure can be applied by placing a weight on the joined layers of the composite. The amount of weight should not be excessive such that the material is damaged by the application of pressure. Appropriate pressure ranges from about 40 kilograms to about 2000 kilograms per square meter. Heat, generally no more than about 400° C., may or may not be applied while the pressure is applied. Pressure generally can be applied for 12 to 48 hours. Gentle heating may accelerate the joining of the layers while pressure is being applied. The composite matrix can be kept moist during the fusing process, or the pressure can be applied while the composite matrix is subjected to dehydrating conditions. The composite matrix can be fully rehydrated after fusing the layers if they are dehydrated or partly dehydrated during the fusing process.

To perform the joining of the layers with chemical crosslinking, the assembled composite matrix structure is placed within a crosslinking solution, generally an aqueous solution. The crosslinking solution permeates through the matrix material to chemically link proteins between layers. If the individual layers are not crosslinked prior to joining the layers, the materials within the layers can also be crosslinked during this process. In preferred embodiments, the composite matrix following crosslinking remains flexible. Suitable crosslinking to obtain flexible matrix material can be accomplished, for example, by using dilute glutaraldehyde solutions or size selected glutaraldehyde, as described above.

In some embodiments, one or more layers are associated with viable cells prior to assembly of the composite. For these embodiments, the assembly approach should be selected to maintain the viability of at least many of the cells following of the composite. For example, surgical adhesives would generally not be significantly toxic to the cells.

Assembly of Composites into Valved Prostheses

The composite tissue matrices can be assembled into a variety of medical devices once the layers are joined together. While various prostheses, as described above, can be produced from the composite materials, there is particular interest in generating valved prostheses. Valved prostheses formed from tissue matrices have flexible leaflets extending across the lumen of the valve. A leaflet support structure provides the framework for the support of the leaflets.

The different components of a prosthesis can incorporate one or more composite tissue matrices and, optionally, additional synthetic materials. The cutting of the composite matrix material can be performed before or after treatment with appropriate compositions for modifying the matrix properties and preparing the matrix for cell colonization. Similarly, the cutting can be performed before or after cell colonization. The assembly of the prosthesis components, if required, also can be performed before or after treatment of the composite matrix and before or after cell colonization.

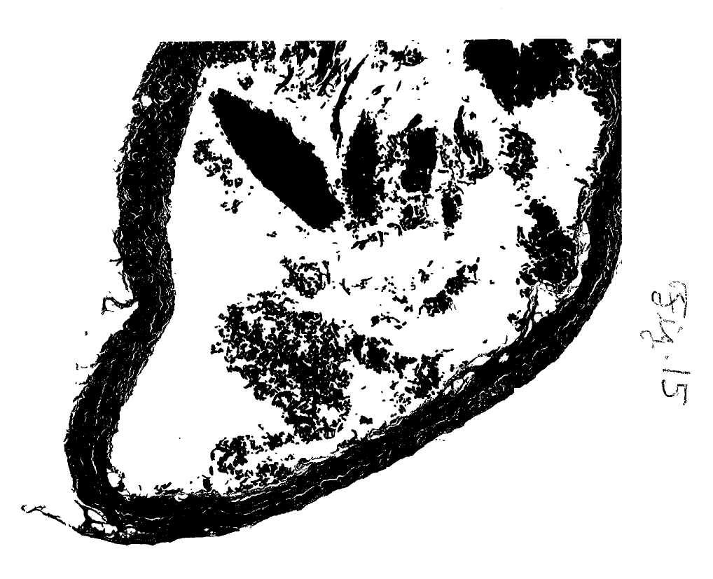

As an example, the heart valve prosthesis of FIG. 1 can be assembled from three structures of tissue portions, as shown in FIGS. 8-10. Each tissue structure can be formed from a composite matrix. If multiple tissue portions are formed from a composite matrix, the composite matrix for one structure may or may not be the same as the composite matrix of another structure within the same prosthesis. Referring to FIG. 8, three leaflet segments 250 are used to form valve 101 (FIG. 1). One leaflet segment 250 forms each of the leaflets 103, 105, 107 in the completed valve 101. Each leaflet segment 250 includes a rounded portion 252, ears 254 and a free edge 256 extending between ears 254.

Referring to FIG. 9, post segments 109 include rectangular tissue segments 260 with a slit 262. Slit 262 is placed over two adjacent leaflets with ears 254 of the two leaflets joined at post segment 109. Once the three leaflets are attached with three post segments 109, free edges 256 of the leaflets extend between post segments 109. By attaching ears 254 to post segment 109, post segment 109 reinforces a commissure post of the valve.

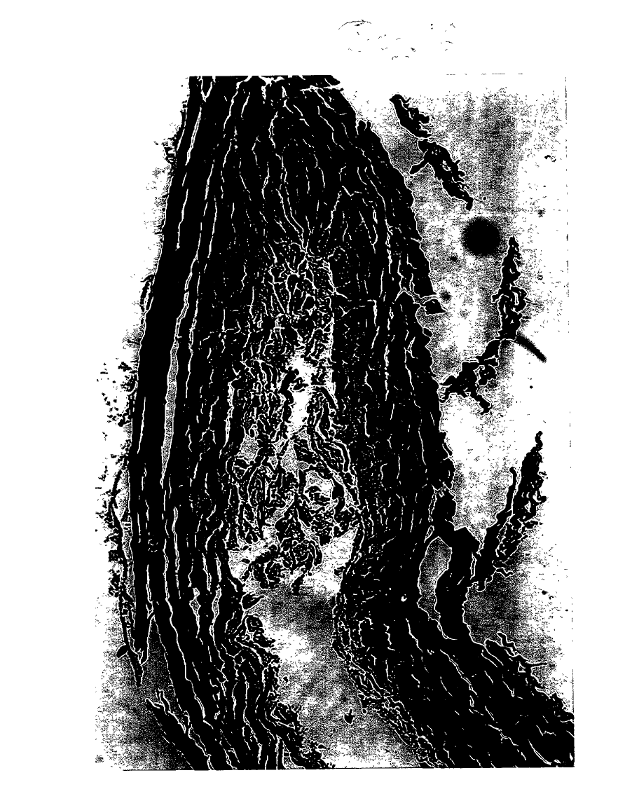

Referring to FIG. 10, rim strip 117 includes curved scalloped sections 264, 266, 268 joined by post sections 270, 272, 274. Scalloped sections 264, 266, 268 are joined to the three respective rounded portions 252 of the three leaflets segments 250. Once joined to the leaflet segments 250, scalloped sections 264, 266, 268 form inflow edge 125 of the valve. Post sections 270, 272, 274 join with post segments 109 and ears 254. Thus, leaflet segments 250 are secured along all of their edges except for free edges 256. Ends 276, 278 of rim strip 117 are secured along a leaflet segment such that rim strip 117 is attached along the circumference of valve 101. Aortic valve prosthesis 101 can be implanted into a patient with a single suture line for faster implantation.

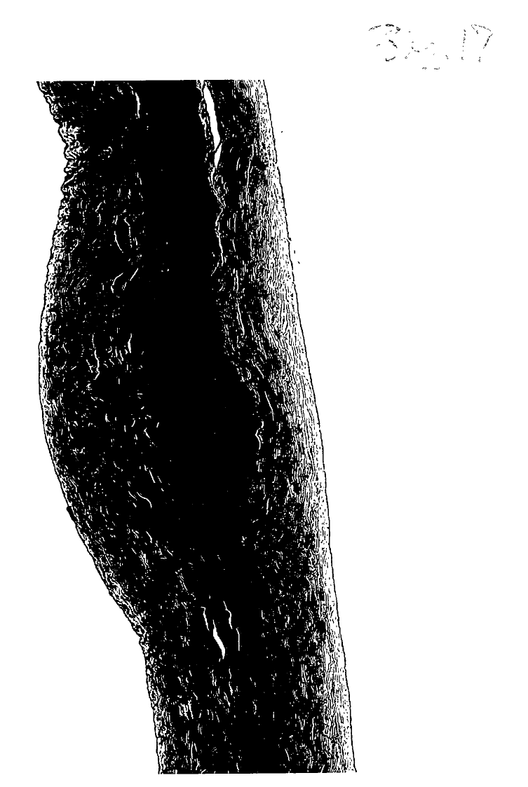

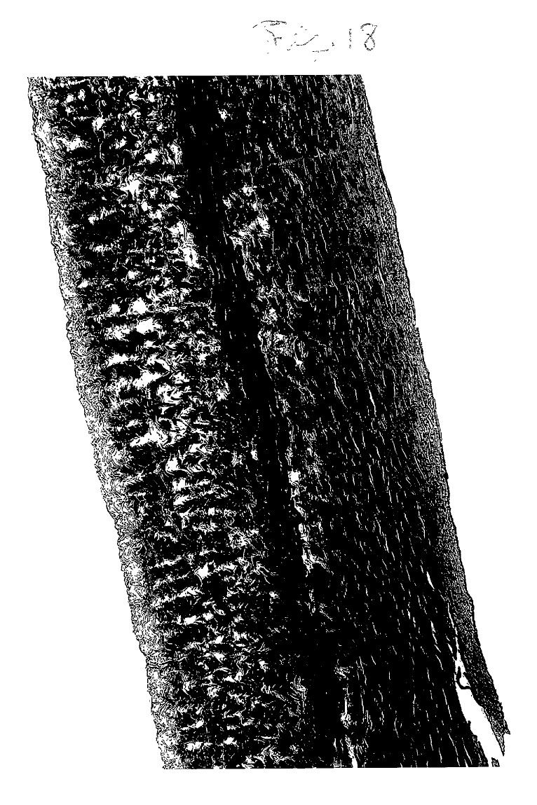

Similarly, the four-leaflet heart valve prosthesis of FIG. 2 can be assembled from four composite matrix components that are joined together to form the valve. These components are shown in FIGS. 11-14. Referring to FIGS. 11-14, leaflet sections 200, 202, 204, 206 each have a section corresponding to one of leaflets 104, 106, 108, 110, respectively. Leaflet sections 200, 202, 204, 206 further include edge sections 210, 212, 214, 216, respectively. Edge sections 210, 212, 214, 216 together form edge 116 that is secured to the sewing ring by insertion between portions 118 and 120 of sewing ring 102, as shown in FIG. 2.

Referring to FIGS. 11-14, folds 218 separate edge sections 210, 212, 214, 216 from leaflets 104, 106, 108, 110. Specifically, leaflets 104, 106, 108, 110 are formed between folds 218 and chordae 112. Slits 220 are cut in leaflet sections 202, 206 to form chordae 112. Similarly, slots 222 are cut in leaflet sections 200, 204 to form chordae 112. Attachment sections 114 extend from the bottom of chordae 112. Additional structures, such as tabs 224, can be included to facilitate assembly of the prosthesis.

To assemble the composite matrix components, leaflet sections 200, 202, 204, 206 are attached to adjacent leaflet sections. Attachment sections 114 are secured into two groupings with one of the two attachment sections 114 of leaflet sections 202, 206 being attached to each group. Chordae 112 remain unattached to decrease interference with blood flow. Edge sections 210, 212, 214, 216 are attached to a sewing ring, as shown in FIG. 2. Attachment can be performed with suture or other attachment approaches. Assembly of a similar valve prosthesis is described U.S. Pat. No. 5,415,667 to Frater, entitled “Mitral Heart Valve Replacement,” incorporated herein by reference.

Colonization of the Tissue with Cells

Some embodiments of the composite matrix are particularly suitable for in vivo or in vitro affiliation of cells with the composite matrix, although the composite matrix can be useful in some applications even if no cell colonization takes place. For in v affiliation with cells following implantation, the matrix material is assembled into a desired medical device and implanted. Since the composite matrix generally is prepared for cell colonization, the matrix is suitable seeding ground for cell colonization by cells that are circulating in the patient's fluids. Thus, circulating cells of the patient affiliate with the composite matrix and can form a repopulated biosynthetic tissue material.

In vitro cell colonization is performed in a cell culture system. With in vitro colonization, the cell colonization can be performed prior to or after assembly of the composite matrix material into a medical device. In some embodiments, a combination of in vivo and in vitro cell colonization can be used. For example, inner layers of the composite can be colonized by selected cells in vitro to provide cell proliferation within the composite while the outer layers of the composite can be colonized m vivo This can lead to a much more rapid spread of cells through the thickness of the composite matrix.

The in vitro affiliation of cells with the composite matrix involves placing the composite matrix into a cell culture system with the desired cells. The cell culture system can include one or more different cell types. Alternatively, the composite matrix can be transferred sequentially to different cell culture systems, each with one or more cell types, for the association of the matrix with multiple cell types. To reduce the possibility of transplant rejection, the mammalian cells used for in vitro colonization preferably are autologous cells, i.e., cells from the ultimate recipient. In vitro affiliation of cells with composite matrix preferably is performed at hospitals where the patient's cells can be removed for use in a cell culture system. Appropriate cells include, for example, endothelial cells, fibroblast cells, corresponding precursor cells and combinations thereof. Association of endothelial cells is particularly appropriate in the production of prostheses that replace structures that naturally have an endothelial or epithelial cell lining, such as vascular components, cardiovascular structures, portions of the lymphatic system, uterine tissue or retinal tissue. Fibroblasts are capable of a variety of different functions depending on their association with a specific tissue. Myofibroblasts are fibroblasts that express relatively more contractile proteins such as myosin and actin.

The cells can be harvested from the patient's blood or bone marrow. Alternatively, suitable cells could be harvested from, for example, adipose tissue of the patient. The harvesting process can involve liposuction followed by collagenase digestion and purification of microvascular endothelial cells. A suitable process is described further in S. K. Williams, “Endothelial Cell Transplantation,” Cell Transplantation 4:401-410 (1995), incorporated herein by reference and in U.S. Pat. Nos. 4,883755, 5,372,945 and 5,628,781, all three incorporated herein by reference.

Purified endothelial cells can be suspended in an appropriate growth media such as M199E (e.g., Sigma Cell Culture, St. Louis, Mo.) with the addition of autologous serum. Other cell types can be suspended similarly. The harvested cells can be contacted with the substrate in a cell culture system to associate the cells with the composite matrix. Thus, a biosynthetic tissue is formed based on cells from the patient prior to implantation.

A composite matrix can be incubated in a stirred cell suspension for a period of hours to days to allow for cell seeding. Cell seeding provides random attachment of cells that can proliferate to line the surface of the prosthetic substrate either before or after implantation into the patient. Alternatively, the composite matrix can be incubated under a pressure gradient for a period of minutes to promote cell sodding. A suitable method for cell sodding can be adapted from a procedure described for vascular grafts in the S. K. Williams article, supra.

In addition, the composite matrix can be placed in a culture system where the patient's cells, such as endothelial cells, are allowed to migrate onto the surface of the prosthetic substrate from adjacent tissue culture surfaces. If either attachment or migration of endothelial cells is performed under conditions involving physiological shear stress, then the endothelial cells colonizing the surface of the substrate may express appropriate adhesion proteins that allow the cells to adhere more tenaciously following implantation.

Similarly, in vitro cell colonization can be performed with an individual layer or an assembled set of layers prior to assembly of the complete composite. The colonization of one or more layers can be performed comparably to the colonization of the composite described above.

Storage, Shipping and Use

The composite matrix can be stored prior to or after formation into a prosthesis. Preferred storage techniques minimize the risk of microbial contamination. For example, the composite matrix can be stored in a sealed container with sterile buffer, saline solution and/or an antimicrobial agent, such as glutaraldehyde or alcohol. Special storage procedures generally are desirable if the composite matrix is colonized with viable cells in vitro in order to maintain cell viability. Specifically, the cellularized tissue can be kept in contact with appropriate growth media.

For distribution, the composite matrix generally is assembled into a prosthesis. The prostheses generally are placed in sealed and sterile containers for shipping. To ensure maintenance of acceptable levels of sterility, the tissue can be transferred to the sterile container using accepted aseptic protocols. The containers can be dated such that the date reflects the maximum advisable storage time.

The containers generally are packaged with instructions for the use of the medical devices along with desired and/or required labels. The containers are distributed to health care professionals for surgical implantation of the prostheses. The implantation is performed by a qualified health care professional. The surgical implantation generally involves the replacement or supplementation of damaged tissue with the prosthesis.

For in vitro cell colonization, the colonized matrix material can be kept under cell culture conditions prior to implantation of the biosynthetic tissue. Preferably, the cell colonization is performed at a medical facility shortly prior to performing the implantation. The cells can be obtained from the ultimate recipient such that rejection will not occur. For these embodiments, the composite matrix prepared for cell colonization is distributed to the medical facilities in appropriate sterile containers.

### EXAMPLES

### Example 1

Composites with Layers of Elastin or Hyaluronic Acid

This example demonstrates the formation of a composite matrix with a layer of flexibility modifying bio-macromolecules/agents within a folded sheet of pericardium.

Sheets of bovine pericardium were obtained from a FDA approved abattoir. The sheets were cleaned of adipose tissue and rinsed in 0.9% saline solution. Nine pericardium sections of approximately 10 cm² each were prepared.

For the preparation of three samples in triplicate, a 2 ml volume of 50% by weight solution/slurry of elastin from bovine neck ligament (Sigma Chemical, St. Louis, Mo.) was applied to one side of each pericardium section at the center and then folded over. For the preparation of three additional samples, a 2 ml volume of 50% by weight solution/slurry of hyaluronic acid (Sigma Chemical, St. Louis, Mo.) was applied to one side of each of the three pericardium sections and then folded over. The folded pericardium sections enveloped the respective solution/slurry within the pericardium. The remaining three percardium sections were folded with no additional material.

The folded samples were subjected to some predrying with a vacuum to carefully avoid bubble formation. Then, the folded samples were lyopholized to dryness. The dried samples were rehydrated and crosslinked for seven days with a solution of 0.5 volume percent glutaraldehyde (EM Science, Cincinnati, Ohio) and 55 mM HEPES buffered saline.

Following crosslinking, one half of each sample was removed and stained with Movat's pentachrome for histological examination. Micrographs were prepared by Histoserv Inc. (Gatherburg, Md.).

A micrograph for the sample prepared with elastin is shown in FIG. 15. Voids are observable in the elastin layer suggesting that the addition of collagen within the elastin layer may help to obtain a layer with fewer or no voids. Some incorporation of the elastin with the pericardium is visible at the edges.

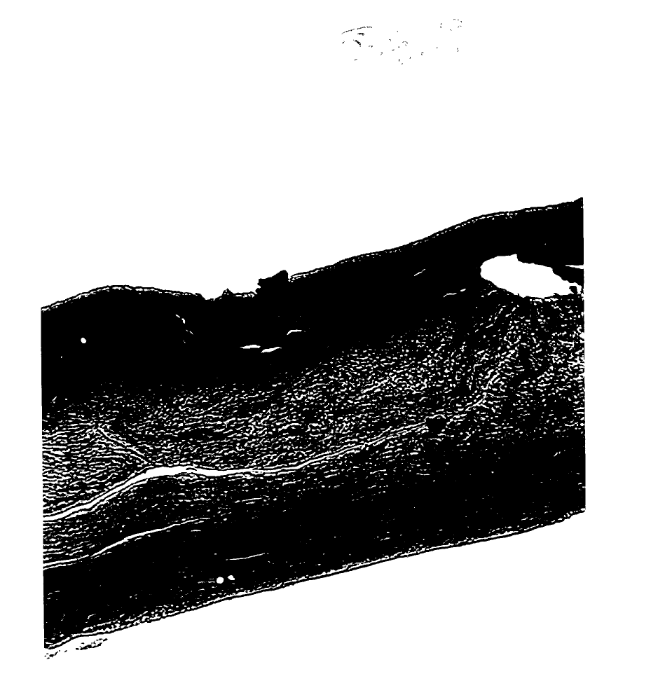

A micrograph for the sample prepared with hyaluronic acid is shown in FIG. 16. Some voids are still visible with these materials, although they are less pronounced than the voids seen in FIG. 15. Considerably more incorporation of the hyalurohic acid into the pericardium is visible at the edges.

### Examples 2

Composites with Proteoglycans and Elastin in a Multi-Layered Collagen Structure

This example involves an evaluation of composites that incorporate proteoglycans or elastin within layers of a collagen structure. The collagen helps to fill voids within the structure.

Sheets of bovine pericardium were obtained from a FDA approved abattoir. The sheets were cleaned of adipose tissue and rinsed in 0.9% saline solution. Six sections of pericardium each with an area of about 10 cm² were prepared.

A first solution was prepared with 5.0 mg/ml of elastin (bovine neck ligament, Sigma Chemical Co., St. Louis, Mo.). A second solution was prepared with 3.0 mg/ml of Vitrogen 100 (collagen, Cohesion, Palo Alto, Calif.). A third solution was prepared with 5.0 mg/ml hyaluronic acid (Sigma Chemical, St. Louis, Mo.). One ml of the elastin solution and 4 mls of the collagen solution were added each on one side of three sections of pericardium. Each sheet was folded to form a layer of the elastin/collagen blend within the pericardium. Also, a one ml quantity of the hyaluronic acid solution and 4 mls of the collagen solution were added each to one side of three additional sections of pericardium. Each sheet was folded to form a layer of hyaluronic acid/collagen blend within the pericardium. The folded sheets were placed in saline buffered to pH 7.4 within a storage container, and stored at 37° C. for 24 hours to allow for repolymerization of the collagen.

After removing the folded sheets from the storage containers, the sheets were lyopholized to dryness. The vacuum was applied slowly to avoid bubble formation in the gelatinous internal component. Then, the samples were rehydrated and crosslinked for 7 days with 0.5% by weight glutaraldehyde (EM Science) solution with 55 mM HEPES buffered saline. One half of each folded sheet was processed for histological examination by staining with Movat's pentachrome and H&E stain. The histological analysis was performed by HistoServ Inc., Gatherburg, Md.

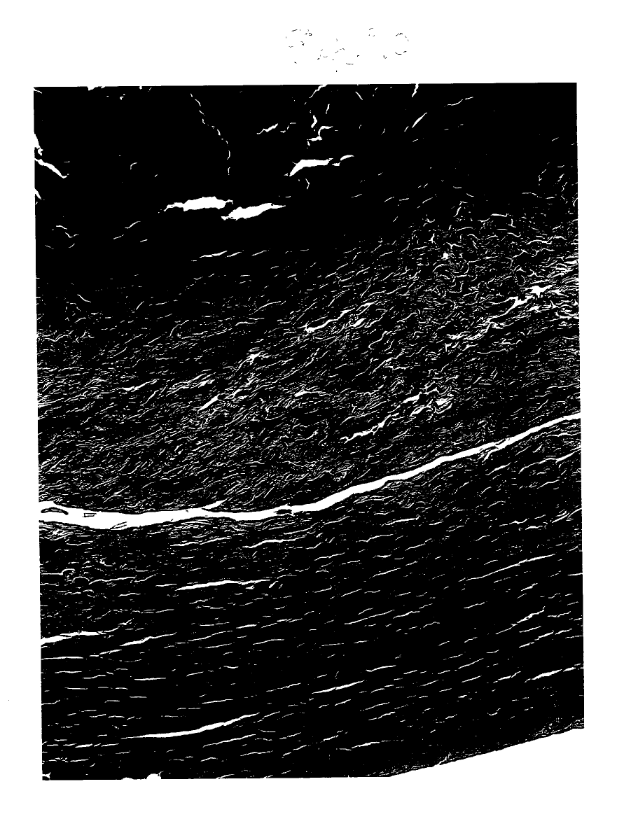

A micrograph of the composite with the elastin/collagen layer within the pericardium is shown in FIGS. 17 and 18. FIG. 17 shows a section through the composite cutting through the bundles of elastin fibers, which are oriented within the central layer of the composite. FIG. 18 shows a cut through the composite along the orientation of the elastin fibrils, which are visible as elongated objects in the center of the figure. The structure forms an integrated network with few voids within the structure. The composite is relatively uniform.

A micrograph of the composite with the hyaluronic acid/collagen layer within the pericardium is shown in FIGS. 19 and 20. Some mixing of the materials at the boundaries between the two layers can be seen in the figures, although distinct layers remain. While some voids can be seen, the materials are relatively uniform with few voids.

The embodiments above are intended to be illustrative and not limiting. Additional embodiments are within the claims. Although the present invention has been described with reference to preferred embodiments, workers skilled in the art will recognize that changes may be made in form and detail without departing from the spirit and scope of the invention.

What is claimed is:

1. A composite matrix comprising a first layer having at least about 5 dry weight percent flexibility modifying agent and a second layer having at least about 5 dry weight percent less flexibility modifying agent than the first layer, wherein at least one layer comprises a reconstituted composition.

2. The composite matrix of claim 1 wherein the second layer has at least about 60 dry weight percent collagen.

3. The composite matrix of claim 1 wherein the second layer has at least about 85 dry weight percent collagen.

4. The composite matrix of claim 1 wherein the second layer comprises crosslinked collagen.

5. The composite matrix of claim 1 wherein the second layer comprises intestinal collagen.

6. The composite matrix of claim 1 wherein the flexibility modifying agent comprises flexibility modifying bio-macromolecules.

7. The composite matrix of claim 6 wherein the flexibility modifying bio-macromolecules comprise an elastic protein.

8. The composite matrix of claim 7 wherein the elastic protein comprises elastin.

9. The composite matrix of claim 1 wherein the first layer has from about 5 to about 95 dry weight percent flexibility modifying agent.

10. The composite matrix of claim 1 wherein the first layer comprises at least about 5 dry weight percent collagen.

11. The composite matrix of claim 1 wherein the flexibility modifying agent comprises friction reducing macromolecules.

12. The composite matrix of claim 11 wherein the friction reducing macromolecules comprise proteoglycans.

13. The composite matrix of claim 11 wherein the friction reducing macromolecules comprise chondroitin sulfate, hyaluronic acid, derivatives thereof or mixtures thereof.

14. The composite matrix of claim 11 wherein the first layer comprises from about 25 to about 90 dry weight percent friction reducing macromolecules.

15. The composite matrix of claim 11 wherein the first layer comprises from about 10 dry weight percent collagen to about 75 dry weight percent collagen.

16. The composite matrix of claim 1 wherein the flexibility modifying agent comprises elastic proteins and friction reducing macromolecules.

17. The composite matrix of claim 1 wherein the second layer has a thickness of at least about 25 microns.

18. The composite matrix of claim 1 wherein the first layer has a thickness of at least about 25 microns.

19. The composite matrix of claim 1 wherein the first layer and the second layer each have a thickness from about 75 microns to about 1 millimeter.

20. The composite matrix of claim 1 wherein the first layer is crosslinked.

21. The composite matrix of claim 1 wherein the first layer is adjacent the second layer.

22. The composite matrix of claim 1 further comprising a third layer having at least about 60 dry weight percent collagen wherein the first layer is between the second layer and the third layer.

23. The composite matrix of claim 22 wherein the first layer comprises viable cells.

24. The composite matrix of claim 1 wherein the second layer is folded over a central core of the first layer.

25. The composite matrix of claim 1 wherein the second layer comprises growth factors.

26. The composite matrix of claim 1 wherein the second layer comprises attachment compounds for fibroblast precursor cells or for vascular endothelial precursor cells.

27. The composite matrix of claim 1 further comprising viable cells.

28. The composite matrix of claim 1 wherein the flexibility modifying agent comprises a synthetic polymer.

29. A valved prosthesis comprising a wall and a plurality of flexible leaflets supported by the wall, the wall comprising a composite matrix having a first layer with at least about 60 dry weight percent collagen and a second layer with at least about 25 dry weight percent collagen and at least about 5 dry weight percent elastin, and the leaflets comprising a composite matrix having a first layer with at least about 60 dry weight percent collagen and a second layer with at least about 25 dry weight percent collagen and at least about 5 dry weight percent proteoglycans.

30. The valved prosthesis of claim 29 wherein the valved prosthesis is a heart valve.

31. The valved prosthesis of claim 30 further comprising chordae.

32. The valved prosthesis of claim 29 wherein the plurality of leaflets is three leaflets.

33. The valved prosthesis of claim 29 wherein the valved prosthesis is a vascular graft.

34. The valved prosthesis of claim 29 further comprising a stent connected to the wall.

35. The valved prosthesis of claim 29 further comprising growth factors.

36. A method of forming a composite matrix, the method comprising fastening a first layer with a second layer, the first layer comprising at least about 25 weight percent collagen and the second layer comprising a flexibility modifying agent.

37. The method of claim 36 wherein fastening the first layer and the second layer comprises applying an adhesive at the interface between the first layer and the second layer.

38. The method of claim 36 wherein the fastening the first layer and the second layer and the second layer comprises applying pressure.

39. The method of claim 36 wherein the fastening the first layer and the second layer and the second layer comprises applying heat.

40. The method of claim 36 wherein the fastening the first layer and the second layer and the second layer comprises chemical crosslinking.
